(19) United States
(12) Patent Application Publication
(10) Pub. No.: US 2026/0090986 A1
(43) Pub. Date: Apr. 2, 2026

(54) CAPSULE INHALER FOR THE ADMINISTRATION OF A PHOSPHODIESTERASE-4 INHIBITOR

(71) Applicant: CHIESI FARMACEUTICI S.P.A., Parma (IT)

(72) Inventors: Francesca BUTTINI, Parma (IT); Giada VARACCA, Parma (IT); Romina OSELLO, Parma (IT)

(73) Assignee: CHIESI FARMACEUTICI S.P.A., Parma (IT)

(21) Appl. No.: 19/112,650

(22) PCT Filed: Sep. 21, 2023
(86) PCT No.: PCT/EP2023/076013

§ 371 Date: Mar. 18, 2025

(30) Foreign Application Priority Data
Sep. 22, 2022 (EP) 22197238.3

(51) Int. Cl. A61K 9/00; A61K 31/4425; A61M 15/00; A61M 15/08

(52) U.S. Cl. CPC A61K 9/0075; A61K 31/4425; A61M 15/0021; A61M 15/004; A61M 15/08

(57) ABSTRACT

The present invention relates to a drug product comprising a single-dose dry powder inhalation device and a pharmaceutical composition filled in a capsule, the pharmaceutical composition comprising micronized particles of the compound of formula (I) and a carrier. The present invention also relates to a drug product or a pharmaceutical composition for use for the treatment of a respiratory disease and to a method for the treatment of a respiratory disease.

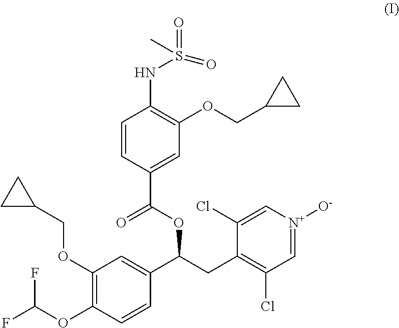 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]

### FIELD OF THE INVENTION

The present invention relates to a drug product comprising a single-dose dry powder inhalation device and a pharmaceutical composition filled in a capsule, the pharmaceutical composition comprising micronized particles of the compound of formula (I) and a carrier. The present invention also relates to a drug product or a pharmaceutical composition for use for the treatment of a respiratory disease and to a method for the treatment of a respiratory disease.

### BACKGROUND OF THE INVENTION

The compound of formula (I)

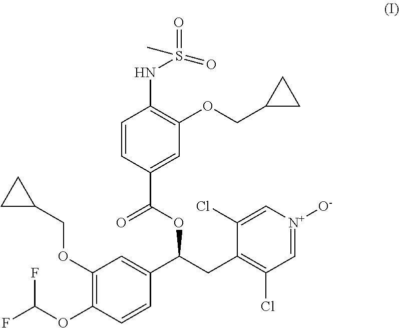 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]

also named tanimilast or CHF6001 or CHF-6001, with INN (3,5-dichloro-4-[(2S)-2-[3-(cyclopropylmethoxy)-4-(difluoromethoxy)phenyl]-2-{[3-(cyclopropylmethoxy)-4-(methanesulfonamido)benzoyl]oxy}ethyl]pyridine1-oxide), is an highly potent and selective PDE4 inhibitor with robust anti-inflammatory activity, currently under clinical development.

Compound of formula (I) has been disclosed in prior art documents in the name of Chiesi: WO 2009/018909 directed to its general formula, methods of preparation, compositions and therapeutic use; WO 2010/089107 specifically directed to sulphonamido derivatives as (−) enantiomers, including compound of formula (I), methods of preparation, compositions and therapeutic use; WO 2012/016889 directed to dry powder compositions comprising the compound of formula (I); WO 2015/059050 directed to crystal form, named Form A, of the compound of formula (I) characterized by specific XRPD peaks and the process for obtaining it.

As other members of the pharmacological class of PDE4 inhibitors, said drug may be indicated for the treatment of lung diseases such as asthma, chronic obstructive pulmonary disease (COPD), bronchiectasis, chronic bronchitis, pneumonia, acute respiratory distress syndrome (ARDS), pulmonary emphysema, smoking-induced emphysema and cystic fibrosis.

Due to well-known systemic side effects associated to the class of PDE4 inhibitors, tanimilast is under development as a composition for inhalation. In fact, one of the advantages of the inhalatory route over the systemic one is the possibility of delivering the drug directly at site of action, avoiding any systemic side-effects.

Currently, tanimilast is in an advanced clinical stage testing two different unitary nominal doses, ie 400 and 800 μg, The product is in the form of a powder composition exploiting the platform technology disclosed in WO 2012/016889 and is administered through the proprietary multidose Nexthaler® inhaler. Said product is indicated hereinafter as the “Reference Product”.

As a carrier, a fissured coarse lactose and a fraction constituted of a mixture of fine lactose and magnesium stearate as a ternary agent are used. Said composition, as disclosed in WO 2012/016889, is indicated hereinafter as the “Reference Composition”.

Thanks to the property of both the inhaler and the platform technology, the composition provides an excellent respirable fraction as well as a significant amount of extrafine particles. There is indeed consensus about the fact that extrafine particles are capable of reaching the distal tract of the respiratory tree, and hence improving small airways outcomes and associated control in the patients affected by the small airways asthma phenotype (Santus P et al, Respir Care 2020; 65(9):1392-1412; Scichilone N et al, Patient Relat Outcome Meas 2014; 5:153-162).

On the other hand, ternary agents are inhaled by the patients and hence add a regulatory burden when seeking approval of the product.

Therefore, it would be advantageous to provide a platform technology for the administration of the compound of formula (I) in the form of a powder, having the same inhalatory performances of the Reference Product, but without the use of ternary agents.

The inventors have surprisingly found that the drug product of the present invention has the same inhalatory performances of the Reference Product, but without the use of ternary agents.

### SUMMARY OF THE INVENTION

In a first aspect, the present invention is directed to a drug product comprising a single-dose dry powder inhalation device, comprising an inhaler body (2) defining a recess (3) for a capsule (4), wherein the capsule (4) holds herein a pharmaceutical composition to be inhaled, a nosepiece or mouthpiece (5) communicating with the recess (3), at least one rupturing element (7) coupled to the inhaler body (2) and configured for rupturing the capsule (4) to allow an outside airflow to be mixed with the pharmaceutical composition of the capsule (4) and inhaled through the nosepiece or the mouthpiece (5), and a pharmaceutical composition filled in a capsule, the pharmaceutical composition comprising micronized particles having a size comprised between 0.1 and 15 micron of the compound of formula (I)

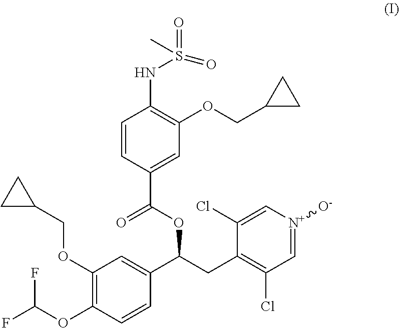 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]

and a carrier consisting of a mixture of coarse and fine particles of pharmaceutically inert acceptable excipient, wherein the inspiratory flow rate of said inhalation device is comprised between 30 l/min and 65 l/min and wherein the carrier consists of a mixture of coarse and fine particles of physiologically acceptable inert excipient in a ratio comprised between 70:30 and 95:5, the coarse particles having an equivalent volume diameter comprised between 200 and 500 micron, while the fine particles having the dv(0.1) comprised between 1 and 5 μm, the dv(0.5) comprised between 18 and 30 μm, and the dv(0.9) comprised between 65 and 95 μm, as measured by means of laser diffraction or sieve analyzer.

In a second aspect, the invention is directed to a pharmaceutical composition according to the invention for use for the treatment of a respiratory disease, wherein said composition is administered using a single-dose dry powder inhalation device, wherein the inspiratory flow rate of said inhalation device is comprised between 30 l/min and 65 l/min.

In a third aspect, the invention provides a method for the treatment of a respiratory disease, the method comprises administering to a patient by inhalation the compound of formula (I), using a drug product as described according to the invention.

In a fourth aspect, the invention is directed to a pharmaceutical composition according to the invention for use for the manufacture of a medicament for the treatment of a respiratory disease, wherein said composition is administered using a single-dose dry powder inhalation device, wherein the inspiratory flow rate of said inhalation device is comprised between 30 l/min and 65 l/min.

In a fifth aspect, the invention provides a process for the preparation of the drug product according to the invention, said process comprising the steps of i) sieving the compound of formula (I) through a suitable mesh, ii) adding the carrier particles to the compound of formula (I), iii) sieving the final blend and mixing to obtain the pharmaceutical composition, iv) filling the obtained pharmaceutical composition in a capsule and v) loading the medicament chamber of the single dry powder inhalation device with the capsule.

### BRIEF DESCRIPTION OF THE FIGURES

FIG. 1: Deposition in the stages of the NGI apparatus of the compound of formula (I) upon delivery of 400 μg Reference Product

FIG. 2: Deposition in the stages of the NGI apparatus of the compound of formula (I) upon delivery of 800 μg Reference Product

FIG. 3: Deposition in the stages of the NGI apparatus of the compound of formula (I) upon delivery of 400 μg drug product of the invention in RS01 high resistance (HR) inhalation device

FIG. 4: Deposition in the stages of the NGI apparatus of the compound of formula (I) upon delivery of 800 μg drug product of the invention in RS01 HR inhalation device

FIG. 5: Comparison of the deposition of the compound of formula (I) upon delivery of 400 μg Reference Product versus 400 μg drug product of the invention in HR RS01 inhalation device

FIG. 6: Comparison of the deposition of the compound of formula (I) upon delivery of 800 μg Reference Product versus 800 μg drug product of the invention in HR RS01 inhalation device

FIG. 7: Comparative in vitro dissolution of the Reference Product and the drug product of the invention (%) at 800 μg unitary nominal dose.

FIG. 8: Comparative in vitro dissolution of the Reference Product and the drug product of the invention (%) at 400 μg unitary nominal dose

FIG. 9: three-dimensional view of a single-dose dry powder inhalation device according to an embodiment of the present invention

FIG. 10: cross section of the single-dose dry powder inhalation device of FIG. 9 in a first operational configuration

FIG. 11: cross section of the single-dose dry powder inhalation device of FIG. 9 in a second operational configuration

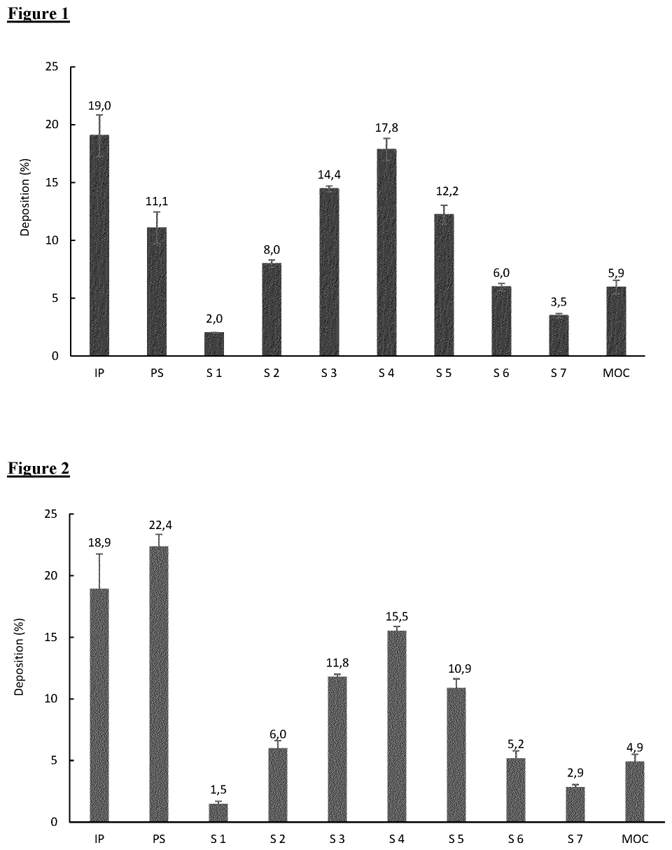

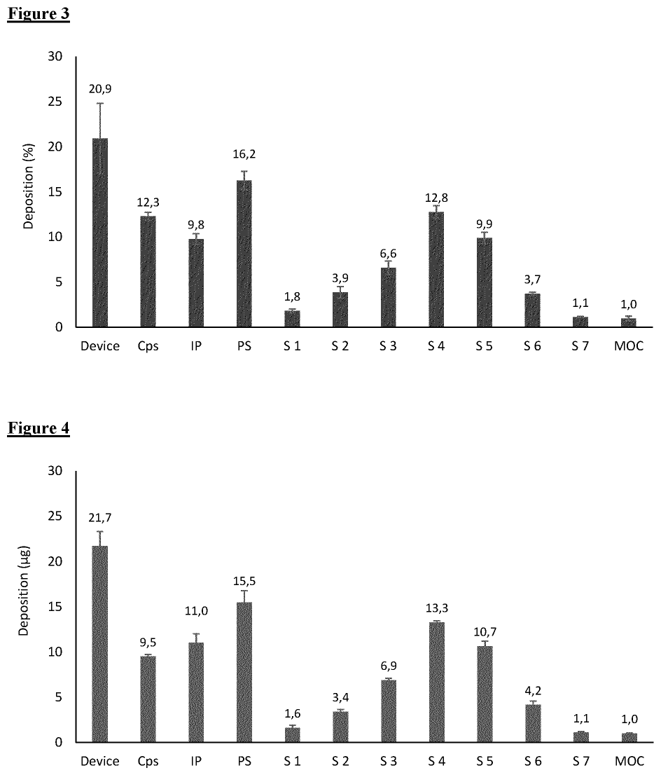

### DETAILED DESCRIPTION OF THE INVENTION

### Definitions

Unless defined otherwise, all technical and scientific terms used herein have the same meanings as commonly understood by the skilled in the art.

Unless otherwise specified, the compound of formula (I) of the present invention is intended to include also polymorphs, stereoisomers, tautomers or pharmaceutically acceptable salts or solvates thereof.

The term “micron”, “micrometers” and μm are used as synonymous.

The term “microgram” and μg are used as synonymous.

The term “percent” and % are used as synonymous.

The term “pharmaceutically acceptable salts”, as used herein, refers to derivatives of compounds of formula (I) wherein the parent compound is suitably modified by converting any of the free acid or basic group, if present, into the corresponding addition salt with any base or acid conventionally intended as being pharmaceutically acceptable. Suitable examples of said salts may thus include mineral or organic acid addition salts of basic residues such as amino groups, as well as mineral or organic basic addition salts of acid residues such as carboxylic groups.

Cations of inorganic bases which can be suitably used to prepare salts comprise ions of alkali or alkaline earth metals such as potassium, sodium, calcium or magnesium.

Those obtained by reacting the main compound, functioning as a base, with an inorganic or organic acid to form a salt comprise, for example, salts of hydrochloric acid, hydrobromic acid, sulfuric acid, phosphoric acid, methane sulfonic acid, camphor sulfonic acid, acetic acid, oxalic acid, maleic acid, fumaric acid, succinic acid and citric acid.

The term “solvate” means a physical association of a compound of this invention with one or more solvent molecules, whether organic or inorganic. This physical association includes hydrogen bonding. In certain instances, the solvate will be capable of isolation, for example, when one or more solvent molecules are incorporated in the crystal lattice of the crystalline solid. The solvate may comprise either a stoichiometric or nonstoichiometric amount of the solvent molecules.

The term “stereoisomer” refers to isomers of identical constitution that differ in the arrangement of their atoms in space. Enantiomers and diastereomers are examples of stereoisomers.

The term “racemate” or “racemic mixture” refers to a composition composed of equimolar quantities of two enantiomeric species, wherein the composition is devoid of optical activity.

The term “tautomer” refers to each of two or more isomers of a compound that exist together in equilibrium and are readily interchanged by migration of an atom or group within the molecule.

The term “composition”, as in pharmaceutical composition, is intended to encompass a product comprising the active ingredient and any pharmaceutically acceptable excipient or carrier, as well as any product which results, directly or indirectly, from combination, complexation or aggregation of any two or more of the ingredients, or from dissociation of one or more of the ingredients, or from other types of reactions or interactions of one or more of the ingredients.

By the term “physiologically acceptable” it is meant a safe, pharmacologically-inert substance utilized as an excipient.

With the term “bioequivalence” it is generally meant the absence of a significant difference between the bioavailability, i.e., the extent of absorption and peak concentration, between two pharmaceutical drug products (e.g., a test product and a reference product) over the course of a period of time, at the same dose and under the same conditions.

The determination of whether or not a test product is bioequivalent to a reference product is determined by performing a study, referred to as a bioequivalence or comparative bioavailability study, in a group of subjects.

For locally acting inhaled products, the term “bioequivalence” is based on more evidence, i.e. similarity of the in vitro test, similarity of systemic exposure and similarity in pharmacokinetic and pharmacodynamic studies to demonstrate equivalence in local delivery.

The term “biowaiver” indicates an exemption granted to a biopharmaceutical company, to show bioequivalence in vivo based on in vitro studies.

The term “vitro-in vivo correlation” (IVIVC) refers to an in vitro dissolution test that is predictive of the in vivo performance of the drug product.

By the term “micronized” it is meant a substance having a size of few microns, typically comprised between 0.1 and 15 micron.

By the term “fine particles” it is meant particles having a size up to few tenths of microns.

By the term “extrafine particles” it is meant particles having a particle size equal or less than 2.0 micron.

The term “coarse” refers to a substance having a size of one or few hundred microns.

In general terms, the particle size of particles is quantified by measuring a characteristic equivalent sphere diameter, known as equivalent volume diameter, by means of laser diffraction or sieve analyzer.

Otherwise, the particle size could be quantified by measuring the mass diameter by means of gravimetric methods, for example utilising suitable known instrument such as the sieve analyser.

The volume diameter (VD) is related to the mass diameter (MD) by the density of the particles (assuming a size independent density for the particles).

In the present application, the particle size of the active ingredients and of fraction of fine and coarse particles is expressed in terms of equivalent volume diameter.

The particles have a log-normal distribution which is defined in terms of the volume or mass median diameter (VMD or MMD) which corresponds to the volume or mass diameter of 50 percent by weight of the particles, and, optionally, in terms of volume or mass diameter of 10% and 90% of the particles, respectively.

Another common approach to define the particle size distribution is to cite three values: i) the median diameter d(0.5) which is the diameter where 50% of the distribution is above and 50% is below; ii) d(0.9), where 90% of the distribution is below this value; iii) d(0.1), where 10% of the distribution is below this value. If said diameter is determined as equivalent volume diameter (the diameter of the hypothetical sphere having the same volume as the particle under examination), the three diameter parameters are indicated as dv(0.5), dv(0.9) and dv(0.1). VMD corresponds to dv(0.5). MMD corresponds to d(0.5).

The span is the width of the distribution based on the 10%, 50% and 90% quantile and is calculated according to the formula.

$$Span = \frac{D [ v , 0.9 ] - D [ v , 0.1 ]}{D [ v , 0.5 ]}$$

In general terms, particles having the same or a similar VMD or MMD can have a different particle size distribution, and in particular a different width of the Gaussian distribution, as represented by the d(0.1) and d(0.9) values.

Upon aerosolisation, the particle size is expressed as mass aerodynamic diameter (MAD), while the particle size distribution is expressed in terms of mass median aerodynamic diameter (MMAD) and Geometric Standard Deviation (GSD). The MAD indicates the capability of the particles of being transported suspended in an air stream. The MMAD corresponds to the mass aerodynamic diameter of 50 percent by weight of the particles.

The terms “additive” and ternary agent” are used as synonymous, and with this term, we mean substances that could modify the detachement of the active ingredient from the surface of the carrier particles.

The term “hard pellets” refers to spherical or semispherical units whose core is made of coarse excipient particles.

The expression “respirable fraction” refers to an index of the percentage of active particles which would reach the lungs in a patient. The respirable fraction, also indicated as Fine Particle Fraction, (FPF), is evaluated using a suitable in vitro apparatus such as Andersen Cascade Impactor (ACI), Multi Stage Liquid Impinger (MLSI) or Next Generation Impactor (NGI), according to procedures reported in common Pharmacopoeias, in particular in the European Pharmacopeia (Eur. Ph.) 11 Edition, paragraph 2.9.18, 372-378. It is calculated by the percentage ratio of the fine particle mass (FPM) (formerly fine particle dose, FPD) to the delivered dose.

The term “peak inspiratory flow rate” refers to the maximal rate of the flow of air during inspiration of the patient through or without the inhalation device.

The term “inspiration flow rate” refers to the constant rate of the flow of air capable to generate a pressure drop across the inhaler of 4.0 kPa (40.8 cm H₂O) during in vitro test in accordance to the European Pharmacopoeia (Eur Ph), 11 Edition, paragraph 0671 Preparations for Inhalation: Inhalanda, 998.

On the basis of the required inspiratory flow rates (l/min) which in turn are strictly depending on their design and mechanical features, DPI's are also divided in:
i) low-resistance devices (about 100 l/min);
ii) medium-resistance devices (about 80 l/min);
iii) high-resistance devices (about 65 l/min);
iv) ultra-high resistance devices (about 40 l/min).

The reported flow rates refer to the pressure drop of 4 kPa (KiloPascal) in accordance to the European Pharmacopeia (Eur. Ph.) 11 Edition, paragraph 0671 Preparations for Inhalation: Inhalanda, 998.

The delivered dose, i.e the amount of drug effectively delivered to the respiratory tree after each actuation of the inhaler, is calculated from the cumulative deposition in the apparatus, while the fine particle mass is calculated from the deposition of particles having a diameter equal or lower than 5.0 micron.

In the context of the present application, the composition is defined as “extrafine” composition when it is able of delivering a fraction of particles having a particle size equal or less than 2.0 micron equal to or higher than 20%, preferably equal to or higher than 25%, more preferably equal to or higher than 30% and/or it is able of delivering a fraction of particles having a particle size equal or less than 1.0 micron equal to or higher than 10%.

The expression “physically stable in the device before use” refers to a composition wherein the active particles do not substantially segregate and/or detach from the surface of the carrier particles both during manufacturing of the dry powder and in the delivery device before use. The tendency to segregate can be evaluated according to Staniforth et al. J. Pharm. Pharmacol. 34,700-706, 1982 and it is considered acceptable if the distribution of the active ingredient in the powder composition after the test, expressed as relative standard deviation (RSD), does not change significantly with respect to that of the composition before the test.

The term “prevention” means the slowing of the progression, delaying the onset, and/or reducing the risk of contracting the disease.

The term “treatment” means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. The term can also mean prolonging survival as compared to expected survival if not receiving treatment.

“Unitary therapeutically effective dose” or “unitary nominal dose” means the quantity of active ingredient to be administered at one time by inhalation upon actuation of the inhalation device. Said dose may be delivered in one or more actuations, preferably one or two actuations (shots) of the inhalation deiv, more preferably in one actuation of the device.

“Daily dose” means the quantity of active ingredient to be administered in a day by inhalation upon actuation of the inhalation device.

“Actuation” refers to the release of active ingredients from the inhalation device by a single activation (e.g. mechanical or breath).

The term “delivered dose” refers to the amount of drug effectively delivered to the respiratory tree after each actuation of the inhalation device.

In the present context, the term “ordered mixture” is referred to the homogeneous composition obtained by admixing the compound of formula (I) of the invention with mixture of pharmaceutically acceptable excipients and/or carriers.

The expression “good homogeneity” refers to a composition wherein, upon mixing, the uniformity of distribution of the active ingredient, expressed as coefficient of variation (CV) also known as relative standard deviation (RSD), is less than 5.0%, preferably equal to or less than 2.5%.

It has been found that it is possible to provide a drug product with the compound of formula (I), wherein the composition gives rise to an in vitro dissolution profile substantially similar to the Reference Product without the use of further additives, so plausibly turning out to be bioequivalent. This is achieved by utilising a single-dose dry powder inhalation device with an inspiratory flow rate comprised between 30 l/min and 65 l/min in combination with an ordered mixture comprising a blend carrier particles having a selected and well-defined particle size.

Furthermore, as reported in Table 10, the drug products according to the invention showed consistent inhalatory parameters independently of the applied flow rate.

As a standard procedure, the inhalatory performances have been determined using an NGI apparatus, testing the compound of formula (I) at the unitary nominal doses of 400 and 800 μg.

Both products according to the invention, in HR RS01 device gave rise to good respirable fractions, i.e. a FPF around 56-58%, with a significant fraction of extrafine particles as well (around 29-30%), as reported in Tables 6 and 7 of Example 3 in the present experimental part, and not substantially different from those of the Reference Product. Both products according to the invention in UHR RS01 device gave rise to good respirable fractions, i.e. a FPF around 53-58%, with a significant fraction of extrafine particles as well (around 27-30%), as reported in Tables 8 and 9 of Example 3 in the present experimental part, and not substantially different from those of the Reference Product.

The comparative analysis of the complete particle size distribution profile of individual stages was also performed according to CPMP/EWP/4151/00, in order to establish the similarity between the Reference Products and the drug products of the invention, which, according to EMA guidelines for biowaivers, shall be considered satisfied if differences are within +/−15%. Although not all the stages satisfied this condition, nevertheless it is well known the inherent variability of multistage impactor/impinger method is rather high, in particular regarding stages wherein a small amount of drug is deposited.

Therefore, an in vitro dissolution system was set up to predict the in vivo performances. The results reported in FIG. 7 and FIG. 8, show an almost overlappable profile for both the drug product of the invention at 400 and 800 μg unitary nominal doses and the Reference Products at the same doses.

Based on these in vitro results and the indications/assumptions of the inhalation Bioclassification System (Hastedt J E et al AAPS/FDA/USP Workshop March 16-17th, Baltimore, AAPS Open, 2016, 2(1), 2016), an IVIV correlation model could be set up to demonstrate an in vitro equivalence and plausibly an in vivo bioequivalence, so candidating the products as a biowaiver.

Advantageously, as it can be appreciated by the plots of FIG. 3 and FIG. 4, for the product of the invention, the fraction of aerosolized drug depositing in the Induction Port (IP) and Pre Separator (PS), mimicking the upper tract of the respiratory tree (Sou T et al J Pharm Sci 2021 110, 66-86), is reduced, thus indicating a lower systemic absorption.

More advantageously, the product of the invention showed consistent values of the aerodynamic parameters at different air flow rate and pressure drop employed in the experiments as reported in Table 10.

In a preferred embodiment of the present invention, with reference to the attached figures, the single-dose dry powder inhalation, which has been generally indicated by the reference number 1, comprises an inhaler body 2 defining a recess 3 for a capsule 4 and a nosepiece or mouthpiece 5 which communicates with the recess 3 and has an opening 6. Two rupturing elements 7 are coupled to the inhaler body 2 and are configured for rupturing the capsule 4 to allow an outside airflow to be mixed with a pharmaceutical composition contained in the capsule 4 and inhaled through the nosepiece or the mouthpiece 5. The two rupturing elements 7 of the single-dose dry powder inhalation device 1 of this embodiment are shaped like pegs or needles and are configured to perforate the capsule 4 when buttons 8 carrying the rupturing elements 7 are pushed and the capsule 4 is located in the recess 3. Air inlets 9 are provided in the inhaler body 2. Said air inlets 9 communicates with the recess 3 to allow the airflow to enter the recess 3 when the user inhales through the nosepiece or mouthpiece 5. A shape and size of the cited air inlets 9 may determine the intrinsic resistance to airflow of the single-dose dry powder inhalation device.

In a preferred embodiment, the present invention provides a drug product comprising a single-dose dry powder inhalation device selected from high-resistance and an ultra-high resistance devices. More preferably, the high resistance device is RS01 with code 239700002AA and the ultra-high resistance device RS01 with code 239700005AA (Plastiape Spa, Osnago, Italy).

The inspiratory flow rate is comprised between 30 l/min and 65 l/min, more preferably between 40 l/min and 65 l/min as referred to the pressure drop of 4 kPa, preferably between 35 l/min and 65 l/min, more preferably between 40 l/min and 65 l/min, even more preferably between 35 l/min and 55 l/min, even more preferably is 65 l/min, even more preferably is 40 l/min.

The unitary nominal dose is comprised between 200 μg and 1000 μg, preferably between 400 μg and 800 μg, more preferably it is 400 μg, even more preferably 800 μg.

Said unitary nominal dose could be delivered in one or more actuations of the inhalation device.

The daily dose at which the pharmaceutical composition comprising the compound of general formula (I) shall be administered is comprised between 800 μg and 4800 μg, preferably between 1200 μg and 3800 μg and more preferably between 1600 μg and 3200 μg.

In one embodiment the daily dose may be reached by a single or double administration.

In another preferred embodiment the daily dose may be reached by a single administration and delivered in one actuation of the inhaler.

In another preferred embodiment the daily dose may be reached by a single administration and delivered in more actuations of the inhaler, preferably two.

In another preferred embodiment the daily dose may be reached by a double administration and delivered in one actuation of the inhaler.

In another preferred embodiment the daily dose may be reached by a double administration and delivered in more actuations of the inhaler, preferably two.

Advantageously, the carrier particles of the invention may be constituted of any physiologically acceptable material or combination thereof, suitable for inhalatory use, so that the preparation of the present composition results in a convenient and versatile process.

For example, said carrier particles may be constituted of one or more materials selected from polyols, for example sorbitol, mannitol and xylitol; crystalline sugars, including monosaccharides and disaccharides; inorganic salts such as sodium chloride and calcium carbonate; organic salts such as sodium lactate; other organic compounds such as urea; polysaccharides, for example starch and its derivatives; and oligosaccharides, for example cyclodextrins and dextrins.

Preferably, said particles are made of a crystalline sugar, even more preferably selected from: a monosaccharide such as glucose or arabinose, or a disaccharide such as maltose, saccharose, dextrose or lactose.

Preferably, said particles are made of lactose, more preferably of alpha-lactose monohydrate.

The carrier consists of a mixture of two distinct fractions, i.e. a fraction of coarse particles and a fraction of fine particles, both made of physiologically acceptable inert excipient, in a ratio comprised between 70:30 and 95:5, preferably between 80:20 and 90:10 by weight. In a preferred embodiment, the ratio is 85:15 by weight. Advantageously the fraction of coarse particles have an equivalent volume diameter comprised between 200 and 500 micrometers. More advantageously, their equivalent volume diameter is comprised between 300 and 480 micrometers, preferably between 350 and 450 micrometers.

Advantageously, the fraction of fine particles have an equivalent volume diameter lower than 100 micron, preferably comprised between 0.5 micron and 99 micron. More advantageously, said particles have the following distribution measured as equivalent volume diameter: the dv(0.1) comprised between 1 and 5 micron, the dv(0.5) comprised between 18 and 30 micron, and the dv(0.9) comprised between 65 and 95 micron.

As it is explained above, the diameter of the particles measured by volume diameter by suitable tools such as laser diffraction or sieve analyzer, could be converted in the equivalent mass diameter knowing the density of the particles.

In a particular embodiment, at least 90% of the particles of the active ingredient have an equivalent volume diameter of less than 6 micron, even more preferably of less than 5 micron. More preferably said particles could have a mean median diameter of 1.8-4 micron.

In another embodiment, the compound of formula (I) has the equivalent volume diameter expressed as dv(0.1) comprised between 0.5 and 1 micron, the dv(0.5) comprised between 1.9 and 2.5 micron, the dv(0.9) comprised between 4 and 6 micron, and the span is comprised between 1.7 and 2.3 micron.

In fact, span values in this range ensure that the population distribution of microparticles is distributed around the diameter median value. Therefore for small values of dv(0.5) (<2.5 μm), there will be in parallel a high portion of extra-fine particles which will favor a peripheral deposition of the drug in the lungs.

The particle size of the compound of formula (I) may be measured by laser diffraction as a dispersion, e.g., using a Mastersizer instrument (Malvern instruments). In particular, the technique is wet dispersion. The equipment is set with the following optical parameters: Refractive index for compound of formula (I)=1.52, Refractive index for dispersant water=1.330, Absorption=1.0 and Obscuration=7-13%. The sample suspension is prepared by mixing approximately 5 mg of sample with 10 ml of water adding 2 drops of Tween 80 in a 25 ml becker. The Dispersion Unit (Malvern instruments) is filled with water and the pump/stirrer in the dispersion unit tank is turned to 3500 rpm and then down to zero to clear any bubbles. The sample suspension is sonicated for 1 minute. The pump/stirrer is turned to 1000 rpm and then the background is measured. Slowly, the prepared suspension sample is dropped into the dispersion unit until a stabilized obscuration of 7-13% is reached, and the analysis started. The analysis was done in triplicate.

According to the present invention the material of the capsules in which is filled the pharmaceutical composition of the present invention is selected from the list comprising, but not limited to, hard gelatin, HPMC, plant-based material, fish gelatin, starch, pullulan, polyvinl acetate (PVA), and soft gelatin. Preferably the capsules are made of HPMC capsules or hard gelatin, or plant-based material.

According to the present invention the capsules in which is filled the pharmaceutical composition of the present invention have a range of sizes comprised between 000 and 5, preferably comprised between 00el and 4, even more preferably comprised between 00 and 3. Even more preferably the capsules has size 2 or 3.

Depending on the chosen inhaler and the required dosage, the skilled person in the art shall select the most suitable size. According to a preferred embodiment of the invention, when RS01 Plastiape inhaler is used, the size of the capsules would be 2 or 3.

According to the present invention, the composition shows an uniformity of distribution of the compound of formula (I), expressed as coefficient of variation (CV) also known as relative standard deviation (RSD), which is less than 5.0%, preferably equal to or less than 2.5%, as shown in Table 3 of Example 2 in the experimental part.

Furthermore the compositions is physically and chemically stable upon storage into the inhaler at room temperature at 60% relative humidity for at least 24 months.

In a preferred embodiment, the present invention provides a drug product comprising a single-dose dry powder inhalation device, comprising an inhaler body (2) defining a recess (3) for a capsule (4), wherein the capsule (4) holds herein a pharmaceutical composition to be inhaled, a nosepiece or mouthpiece (5) communicating with the recess (3), at least one rupturing element (7) coupled to the inhaler body (2) and configured for rupturing the capsule (4) to allow an outside airflow to be mixed with the pharmaceutical composition of the capsule (4) and inhaled through the nosepiece or the mouthpiece (5), and a pharmaceutical composition filled in a capsule, the pharmaceutical composition comprising micronized particles having a size comprised between 0.1 and 15 micron of the compound of formula (I)

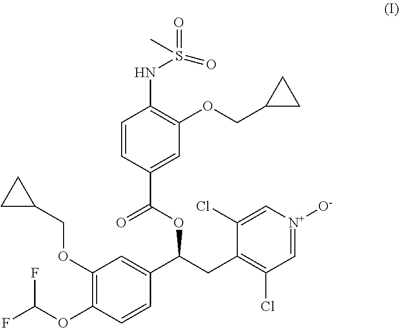 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]

and a carrier consisting of a mixture of coarse and fine particles of pharmaceutically inert acceptable excipient, wherein the inspiratory flow rate of said inhaler is comprised between 30 l/min and 65 l/min and wherein the carrier consist of a mixture of coarse and fine particles of physiologically inert acceptable excipient in a ratio comprised between 80:20 and 90:10, wherein the coarse particles have an equivalent volume diameter comprised between 200 and 500 micron, while the fine particles having the dv(0.1) comprised between 1 and 5 μm, the dv(0.5) comprised between 18 and 30 μm, and the dv(0.9) comprised between 65 and 95 μm, as measured by means of laser diffraction or sieve analyzer.

In another preferred embodiment, the present invention provides a pharmaceutical composition according to the invention for use for the treatment of a respiratory disease, wherein said composition is administered using a single-dose dry powder inhalation device, wherein the inspiratory flow rate of said inhalation device is comprised between 30 l/min and 65 l/min.

In another preferred embodiment, the present invention provides the drug product of the invention, for use for the treatment of an inflammatory or obstructive respiratory disease. As an alternative, the invention provides the pharmaceutical formulation according to the invention, upon administration by the single-dose inhaler according to the invention for use for the treatment of an inflammatory or obstructive respiratory disease.

In a further preferred embodiment, the present invention provides the drug product as defined above, for use for the treatment of an inflammatory or obstructive respiratory disease selected from: asthma, chronic obstructive pulmonary disease (COPD), bronchiectasis, chronic bronchitis, pneumonia, acute respiratory distress syndrome (ARDS), pulmonary emphysema, smoking-induced emphysema and cystic fibrosis.

Although the carrier shall consist of the particles according to the invention, the composition may comprise further active ingredients and, optionally other excipients, for example sweeteners and flavoring agents.

The further active ingredients could be selected from those currently utilized for the prevention and treatment of a respiratory disease by inhalation, for example beta₂-agonists, corticosteroids and anticholinergic agents.

In an even further preferred embodiment, the present invention provides the drug product of the invention as defined above as add-on to a single, double or triple therapy.

In an even further preferred embodiment, the present invention provides the drug product of the invention as defined above as add-on to a single, double or triple therapy.

In another preferred embodiment, the present invention provides the drug product of the invention as defined above wherein the single, double or triple therapy active agents are selected from beta₂-agonists, corticosteroids and anticholinergic agents.

In another embodiment, the invention provides a process for the preparation of a pharmaceutical composition according to the invention, said process comprising the steps of sieving the compound of formula (I) through a suitable mesh, adding the carrier to the compound of formula (I), sieving the final blend and mixing.

In another preferred embodiment, the invention provides a process for the preparation of a pharmaceutical composition according to the invention, said process comprising the steps of sieving the compound of formula (I) through a mesh with one-third of the carrier and mix in a mixer, adding a second-third of the carrier to the blend and mixing, adding the last third of the the carrier and mixing, sieving the blend and mixing.

Then, a capsule is filled with the pharmaceutical composition according to the invention and the capsule is loaded into the medicament chamber of the single dry powder inhalation device.

The filling of the capsule and the loading of the inhaler are performed according to the knowledge of the skilled person in the art.

In another preferred embodiment, the invention provides a process for manufacturing a drug product comprising a step of filling the medicament chamber of a single dry powder inhalation device with capsule filled with a pharmaceutical composition according to the invention.

In an even further preferred embodiment, the present invention provides a method for the treatment of a respiratory disease, the method comprising administering to a patient by inhalation the compound of formula (I), using a drug product as described according to the invention.

In another preferred embodiment, the present invention provides a method as defined above, for treatment of a respiratory disease selected from the above mentioned inflammatory or obstructive respiratory disease.

The invention is also directed to an inhalation device, in form of a single-dose dry powder inhaler loaded with a pharmaceutical composition comprising micronized particles of the compound of formula (I) and a carrier, wherein the inspiratory flow rate of said inhaler is comprised between 30 l/min and 65 l/min and wherein the carrier consist of a physiologically acceptable blend of inert excipient having a coarse fraction of particles with an equivalent volume diameter between 200-500 micron and a fine portion with an equivalent volume diameter lower than 100 micron, as described above according to the invention.

The following non-limiting examples are illustrative for the disclosure and are not to be construed as to be in any way limiting for the scope of the invention.

### EXPERIMENTAL PART

### Abbreviations

MOC=Micro-Orifice Collector; HR=high resistance; IP=Induction Port; PS=Pre Separator; UHR=ultra high resistance

### Examples

### Example 1: Preparation of the Blend Composition with Compound of Formula (I) and The Mixture of Coarse Lactose:Fine Lactose 85:15

The coarse lactose and fine lactose employed for the preparation of the composition were respectively Pharmatose 50 M (dv0.9 of 490 μm) and InhaLac® 150 (dv0.9<95 μm). The coarse lactose was mechanically vibrated and sieved for 30 min and only the particles in the range 355-490 μm were employed for the next steps. The ratio coarse:fine was 85:15. The fine and the coarse carrier were previously mixed for 50 minutes in a Turbula mixer at 38 rpm. Tanimilast was sieved through a 500 μm mesh with one-third of carrier and mixed in a Turbula mixer operating at a rotation speed of 38 r.p.m for 40 minutes. Then, a second-third of the carried was added to the blend and mixed at 38 r.p.m for 40 minutes. Finally, the last part of the carrier was added and mixed at 38 r.p.m for 40 minutes. The resulting mix was forced through 500 μm mesh to remove any agglomerates and mixed for 30 minutes at 38 r.p.m. A 10 grams batches size were produced.

TABLE 1
Composition of the invention, 400 μg

Amount per shot of the inhaler

| Blend components (400 μg) | mg | % |
|---|---|---|
| Compound of formula (I) | 0.4 | 2.0 |
| Coarse lactose | 16.7 | 83.5 |
| Fine lactose | 2.9 | 14.5 |
| Total weight | 20.0 | 100.0 |

TABLE 2
Composition of the invention, 800 μg

Amount per shot of the inhaler

| Blend components (800 μg) | mg | % |
|---|---|---|
| Compound of formula (I) | 0.8 | 4.0 |
| Coarse lactose | 16.3 | 85 |
| Fine lactose | 2.9 | 15 |
| Total weight | 20.0 | 100.0 |

### Example 2: Content of the Compound of Formula (I) (μg)/20 mg of the Composition of The Invention, ±St.Dev and CV % (n=6)

The uniformity of drug content in the blends was determined with HPLC. The analysis was conducted on 6 samples, collected randomly in the mixture, dissolved in 100 ml of acetonitrile/water (60/40) v/v used as solvent. 20 mg were weighed for each sample.

TABLE 3
content of the compound of formula (I) at 400 or 800 μg strenght/20
mg of the composition of the invention, ±St. Dev and
CV % (n = 6)

| | 400 μg of compound of | 800 μg compound of |
|---|---|---|
| Samples | formula (I)/20 mg | formula (I)/20 mg |
| #1 | 402.1 | 802.9 |
| #2 | 393.1 | 792.4 |
| #3 | 388.9 | 806.0 |
| #4 | 407.6 | 804.9 |
| #5 | 405.7 | 810.3 |
| #6 | 380.2 | 798.4 |
| Mean (±St. Dev) | 396.2 ± 10.7 | 802.5 ± 6.3 |
| CV(%) | 2.7 | 0.8 |

The blends in Table 3 show an excellent accuracy and uniformity of distribution (precision as CV) of the active ingredient.

### Example 3: Determination of the Aerodynamic Particle Size Distribution (APSD)

The in vitro aerodynamic assessment was carried out using a Next Generation Impactor (NGI), following the procedure detailed in the European Pharmacopoeia 10.0 in the 2.9.18 “Preparation for inhalation: Aerodynamic assessment of fine particles” chapter at pages 347-360. Nexthaler (Chiesi, Parma, Italy), combined with the composition prepared according to WO 2012/016889 at 400 μg and 800 μg was considered as the Reference Products.

RS01 high resistance with code 239700002AA and RS01 ultra-high resistance with code 239700005AA devices (Plastiape, Osnago, LC Italy) were used to conduct the analysis with the Drug Products according to the composition invention at 400 μg and 800 μg. The capsules used were Quali-V®-I, size 3 (Qualicaps Europe, S.A.U.) and loaded with about 20 mg. The DPI inhalers were activated at a pressure drop of 4 kPa corresponding at a flow rate of 57.5 L/min for Nexthaler, 65 L/min for RS01 high resistance and 40 L/min for RS01 ultra-high resistance for a duration of time sufficient to sample an air volume of 4.0 liters. The NGI was connected to the vacuum pump and the airflow was fixed using a flowmeter. The analysis was performed under critical flow control conditions. The device was connected to the NGI through a rubber adaptor, and one single dose was discharged and collected into the apparatus. The drug remaining in capsule and device (only for RS01 analysis), and the drug deposited in the different portions of the impactor was recovered using acetonitrile/water (60/40) v/v as solvent. The samples were filtered with RC filter (0.45 μm) and quantified using HPLC to determine the amount of drug. In all the experiments carried out, the percentage of active principle recovered in the whole instrument was higher than 85% of the nominal dose.

The metered dose (MD) was calculated by summing the drug recovered from the impactor (IP, PS, stages 1 to 7 and MOC) and the drug remaining in the inhaler (capsule and device). It wasn't possible to quantify MD for the multidose Nexthaler DPI since it is a reservoir multidose inhaler and cannot be wet and rinsed at the end of the experiment.

The Emitted Dose (ED) is the amount of drug leaving the device and entering the impactor and was calculated by summing the drug recovered from the impactor (IP, PS, stages 1 to 7 and MOC).

The drug deposition in the impactor allowed the calculation of the aerodynamic parameters.

The mass median aerodynamic diameter (MMAD) and Geometric Standard Deviation (GSD) were determined by plotting the cumulative percentage of mass less than the stated aerodynamic diameter for each NGI stage on a probability scale versus the aerodynamic diameter of the stage on a logarithmic scale. Linear regression of the six data points closest to 50% of the cumulative particle mass that entered the impactor was performed to compute the MMAD and GSD.

The Fine Particle Mass (FPM) was calculated as the mass of drug <5 μm (calculated from the log-probability plot equation) and the Fine Particle Fraction (FPF) was determined as the ratio between FPD and ED in percent.

The Extra Fine Particle Mass (EFPM) was calculated as the mass of drug below 2 μm (calculated from the log-probability plot equation) and the Extra Fine Particle Fraction (EFPF) was determined as the ratio between EFPD and ED in percent.

TABLE 4
APSD of Nexthaler device loaded with the Reference Composition at 400 μg

| Reference Composition | Device | Device | Device | Mean ± St. | |
|---|---|---|---|---|---|
| (400 μg/20 mg) | #1 | #2 | #3 | Dev | CV(%) |
| Shot weight (mg) | 19.6 | 21.4 | 22.1 | 21.0 ± 1.3 | 6.1 |
| Emitted dose (μg) | 341.3 | 384.3 | 403.9 | 376.5 ± 32.1 | 8.1 |
| MMAD (μm) | 2.12 | 2.04 | 2.07 | 2.08 ± 0.04 | 1.95 |
| GSD | 2.47 | 2.55 | 2.50 | 2.51 ± 0.04 | 1.60 |
| Fine Particle Mass (μg) | 197.4 | 238.6 | 253.3 | 229.8 ± 29.0 | 12.6 |
| Fine Particle Fraction (%) | 57.8 | 62.1 | 62.7 | 60.9 ± 2.6 | 4.3 |
| Extra Fine Particle Mass (μg) | 108.2 | 135.2 | 140.9 | 128.1 ± 17.5 | 13.6 |
| Extra Fine Particle Fraction (%) | 31.7 | 35.2 | 34.9 | 33.9 ± 1.9 | 5.7 |

Shot weight (mg), Emitted Dose (μg), MMAD (μm), GSD, FPM (μg), FPF (%), EFPM (μg), EFPF (%) mean values of Nexthaler device loaded with the Reference Composition (400 μg of CHF001/20 mg) ± St. dev and CV % in (n = 3)

TABLE 5
APSD of Nexthaler device loaded with the Reference Composition at 800 μg

| Reference Composition | Device | Device | Device | Mean ± St. | |
|---|---|---|---|---|---|
| (800 μg/20 mg) | #1 | #2 | #3 | Dev | CV(%) |
| Shot weight (mg) | 20.7 | 21.8 | 20.0 | 20.8 ± 0.9 | 4.4 |
| Emitted dose (μg) | 823.3 | 859.4 | 769.8 | 817.5 ± 45.1 | 5.5 |
| MMAD (μm) | 2.00 | 2.00 | 2.05 | 2.02 ± 0.05 | 1.43 |
| GSD | 2.42 | 2.49 | 2.40 | 2.44 ± 0.05 | 1.92 |
| Fine Particle Mass (μg) | 417.8 | 467.2 | 394.7 | 426.6 ± 37.0 | 8.7 |
| Fine Particle Fraction (%) | 50.8 | 54.4 | 51.3 | 52.1 ± 2.0 | 3.7 |
| Extra Fine Particle Mass (μg) | 234.8 | 265.3 | 217.8 | 239.3 ± 24.0 | 10.0 |
| Extra Fine Particle Fraction (%) | 28.5 | 30.9 | 28.3 | 29.2 ± 1.4 | 4.9 |

Shot weight (mg), Emitted Dose (μg), MMAD (μm), GSD, FPM (μg), FPF (%), EFPM (μg), EFPF (%) mean values of Nexthaler device loaded with the Reference Composition (800 μg of CHF001/20 mg) ± St. dev and CV % in (n = 3)

TABLE 6
APSD of HR RS01 device loaded with the composition of the invention at 400 μg

| Composition of the invention | Device | Device | Device | Mean ± St. | |
|---|---|---|---|---|---|
| (400 μg/20 mg) | #1 | #2 | #3 | Dev | CV(%) |
| Shot weight (mg) | 20.2 | 20.2 | 20.0 | 20.1 ± 0.1 | 0.6 |
| Metered dose (μg) | 398.5 | 382.5 | 387.6 | 389.5 ± 8.2 | 2.1 |
| Emitted dose (μg) | 257.1 | 271.3 | 251.6 | 260.0 ± 10.1 | 3.9 |
| MMAD (μm) | 2.02 | 2.15 | 2.06 | 2.08 ± 0.07 | 3.21 |
| GSD | 2.13 | 2.11 | 2.11 | 2.12 ± 0.01 | 0.59 |
| Fine Particle Mass (μg) | 142.1 | 151.8 | 143.7 | 145.9 ± 5.2 | 3.6 |
| Fine Particle Fraction (%) | 55.3 | 56.0 | 57.1 | 56.1 ± 0.9 | 1.7 |
| Extra Fine Particle Mass (μg) | 76.3 | 77.3 | 75.6 | 76.4 ± 0.8 | 1.1 |
| Extra Fine Particle Fraction (%) | 29.7 | 28.5 | 30.1 | 29.4 ± 0.8 | 2.8 |

Shot weight (mg), Metered dose (μg), Emitted Dose (μg), MMAD (μm), GSD, FPM (μg), FPF (%), EFPM (μg), EFPF (%) mean values of RS01 device loaded with the composition of the invention (400 μg of compound of formula (I)/20 mg) ± St. dev and CV % in (n = 3)

TABLE 7
APSD of HR RS01 device loaded with the composition of the invention at 800 μg

| Composition of the invention | Device | Device | Device | Mean ± St. | |
|---|---|---|---|---|---|
| (800 μg/20 mg) | #1 | #2 | #3 | Dev | CV(%) |
| Shot weight (mg) | 20.1 | 20.4 | 20.1 | 20.2 ± 0.2 | 0.9 |
| Metered dose (μg) | 802.6 | 774.5 | 803.6 | 793.6 ± 16.6 | 2.1 |
| Emitted dose (μg) | 540.2 | 547.1 | 549.0 | 545.4 ± 4.6 | 0.8 |
| MMAD (μm) | 1.95 | 2.08 | 2.02 | 2.02 ± 0.07 | 3.23 |
| GSD | 2.06 | 2.08 | 2.08 | 2.07 ± 0.01 | 0.50 |
| Fine Particle Mass (μg) | 327.4 | 301.5 | 313.1 | 314.0 ± 13.0 | 4.1 |
| Fine Particle Fraction (%) | 60.6 | 55.1 | 57.0 | 57.6 ± 2.8 | 4.9 |
| Extra Fine Particle Mass (μg) | 177.5 | 157.0 | 166.1 | 166.9 ± 10.3 | 6.2 |
| Extra Fine Particle Fraction (%) | 32.9 | 28.7 | 30.3 | 30.6 ± 2.1 | 6.9 |

Shot weight (mg), Metered dose (μg), Emitted Dose (μg), MMAD (μm), GSD, FPM (μg), FPF (%), EFPM (μg), EFPF (%) mean values of RS01 device loaded with the composition of the invention (800 μg of compound of formula (I)/20 mg) ± St. dev and CV % in (n = 3)

As shown in Tables 6 and 7, both products according to the invention in HR RS01 device gave rise to good respirable fractions, i.e. a FPF around 56-58%, with a significant fraction of extrafine particles as well (around 29-30%), and similar to those of the Reference Product, as resulting from the comparison with Tables 4 and 5.

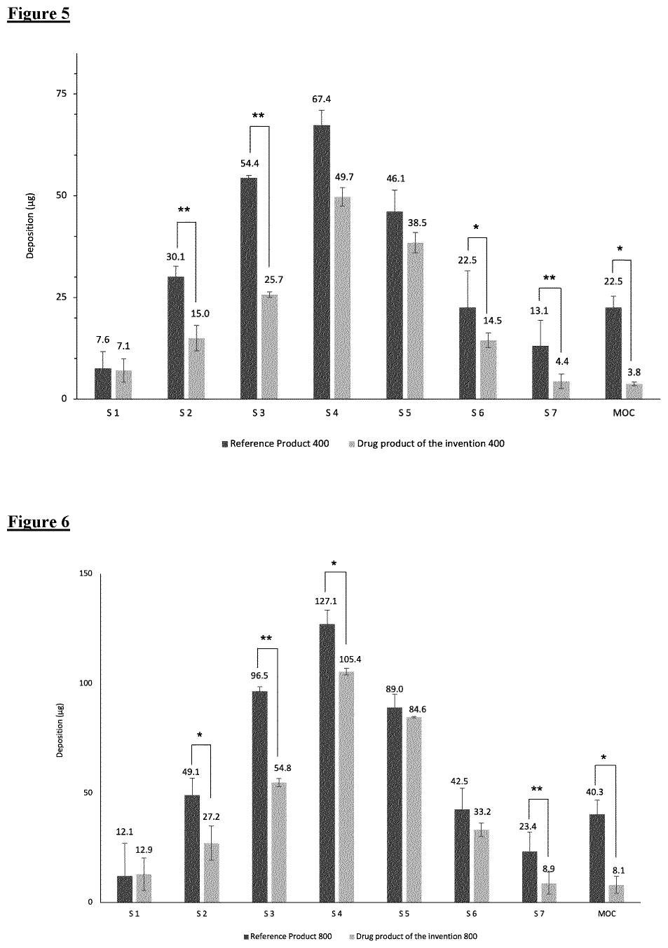

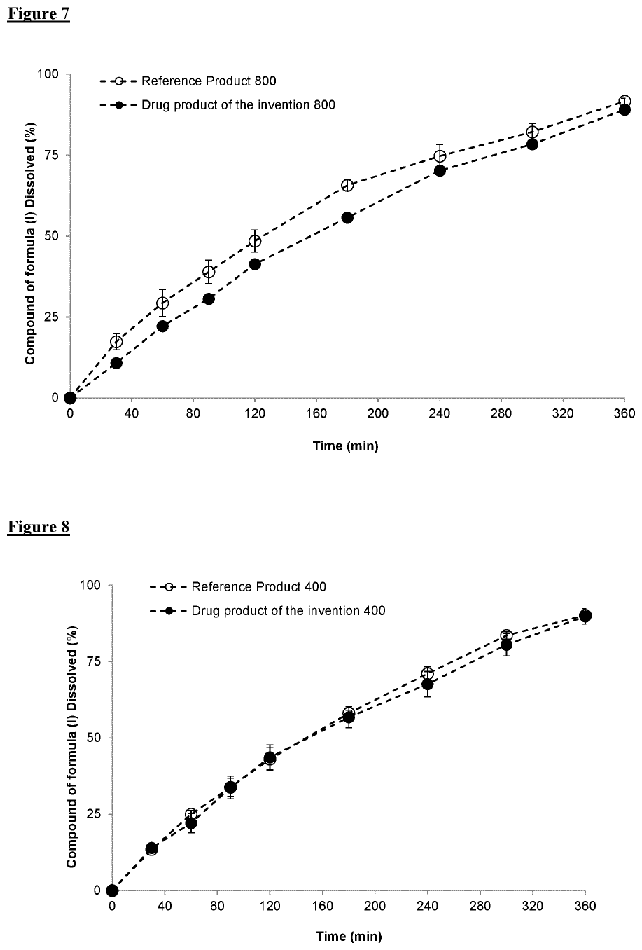

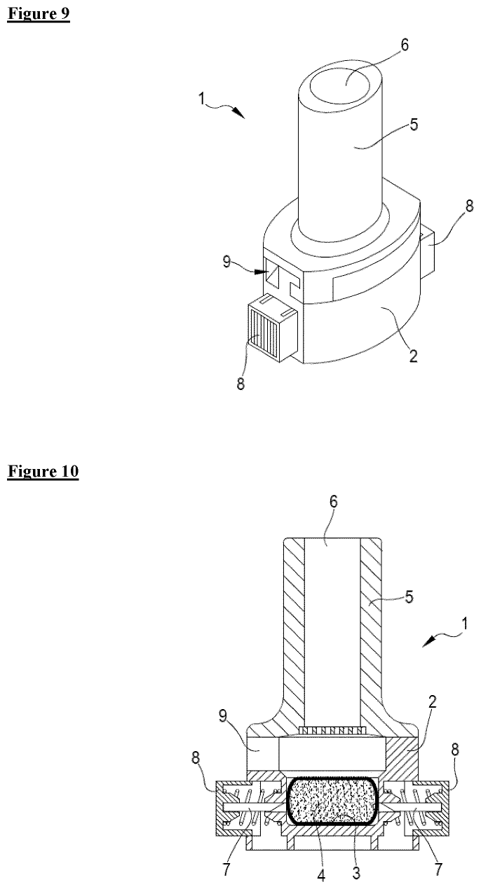

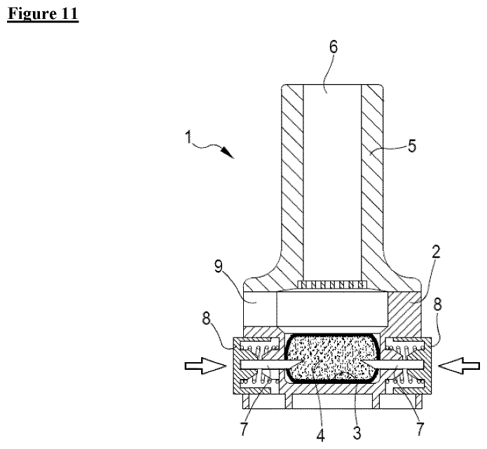

As shown in FIGS. 5 and 6, upon in vitro assessment of the inhalatory performances, the amount of the emitted dose which remains in the first two stages of the impactor mimicking the upper part of the respiratory tree (throat, large bronchi) is lower for the composition of the invention in comparison with the Reference Composition. This would lead to a lower systemic absorption of the API.

TABLE 8
APSD of UHR device loaded with the composition of the invention at 400 μg

| Composition of the invention | Device | Device | Device | Mean ± St. | |
|---|---|---|---|---|---|
| (400 μg/20 mg) | #1 | #2 | #3 | Dev | CV(%) |
| Shot weight (mg) | 19.6 | 19.3 | 19.4 | 19.4 ± 0.2 | 0.8 |
| Metered dose (μg) | 386.0 | 359.5 | 376.2 | 373.9 ± 13.4 | 3.6 |
| Emitted dose (μg) | 279.8 | 221.4 | 268.1 | 256.4 ± 30.9 | 12.0 |
| MMAD (μm) | 2.15 | 2.11 | 2.16 | 2.14 ± 0.03 | 1.24 |
| GSD | 1.89 | 1.93 | 1.89 | 1.90 ± 0.02 | 1.26 |
| Fine Particle Mass (μg) | 148.9 | 115.3 | 145.2 | 136.5 ± 18.5 | 13.5 |
| Fine Particle Fraction (%) | 53.2 | 52.1 | 54.2 | 53.2 ± 1.1 | 1.99 |
| Extra Fine Particle Mass (μg) | 75.1 | 60.1 | 72.7 | 69.3 ± 8.1 | 11.6 |
| Extra Fine Particle Fraction (%) | 26.9 | 27.2 | 27.1 | 27.0 ± 0.2 | 0.6 |

Shot weight (mg), Metered dose (μg), Emitted Dose (μg), MMAD (μm), GSD, FPM (μg), FPF (%), EFPM (μg), EFPF (%) mean values of UHR RS01 device loaded with the composition of the invention (400 μg of compound of formula (I)/20 mg) ± St. dev and CV % in (n = 3)

TABLE 9
APSD of UHR device loaded with the composition of the invention at 800 μg

| Composition of the invention | Device | Device | Device | Mean ± St. | |
|---|---|---|---|---|---|
| (800 μg/20 mg) | #1 | #2 | #3 | Dev | CV(%) |
| Shot weight (mg) | 19.4 | 19.4 | 19.5 | 19.4 ± 0.1 | 0.3 |
| Metered dose (μg) | 814.0 | 806.0 | 810.0 | 810.0 ± 4.0 | 0.5 |
| Emitted dose (μg) | 544.7 | 521.2 | 534.0 | 533.3 ± 11.8 | 2.2 |
| MMAD (μm) | 2.16 | 2.08 | 2.13 | 2.12 ± 0.04 | 1.90 |
| GSD | 1.86 | 1.88 | 1.87 | 1.87 ± 0.01 | 0.52 |
| Fine Particle Mass (μg) | 312.4 | 311.2 | 315.6 | 313.1 ± 2.3 | 0.73 |
| Fine Particle Fraction (%) | 57.3 | 59.7 | 59.1 | 58.7 ± 1.2 | 2.09 |
| Extra Fine Particle Mass (μg) | 155.9 | 161.5 | 155.1 | 159.0 ± 2.9 | 1.8 |
| Extra Fine Particle Fraction (%) | 28.6 | 31.0 | 29.9 | 29.8 ± 1.2 | 4.0 |

Shot weight (mg), Metered dose (μg), Emitted Dose (μg), MMAD (μm), GSD, FPM (μg), FPF (%), EFPM (μg), EFPF (%) mean values of UHR RS01 device loaded with the composition of the invention (800 μg of compound of formula (I)/20 mg) ± St. dev and CV % in (n = 3)

As shown in Tables 8 and 9, both products according to the invention in UHR RS01 device gave rise to good respirable fractions, i.e. a FPF around 53-58%, with a significant fraction of extrafine particles as well (around 27-30%), and similar to those of the Reference Product, as resulting from the comparison with Tables 4 and 5.

### Example 4: Dissolution Test

In vitro dissolution tests were carried out to compare the performance of the Reference Product and the drug product of the invention at 400 μg and 800 μg, using RespiCell™ (EU registration No 006649570-0001). Said apparatus has built-up to overcome some of the shortfalls of the types of dissolution testing currently used for pulmonary products (Sonvico F. et al Pharmaceutics 2021, 13 (10), 1541).

It is a vertical diffusion cell apparatus that comprises a 170 cm³ reservoir filled with the dissolution media and a side arm of 10 cm length. The apparatus is composed of an upper part, the donor chamber, and a lower part, the receptor chamber, linked by a clamp and separated by a glass fiber filter, used as diffusion membrane, and sit horizontally in contact with the dissolution medium. The receptor chamber contains a magnetic stirrer inside it. Type A/E glass fiber filter of 76 mm diameter (PALL Corporation, Port Washington; NY, USA) were employed as diffusion membrane. The dissolution medium employed for the analysis was phosphate-buffer saline (PBS) with 0.5% of Sodium dodecyl sulfate (SDS). RespiCell™ was connected to a heating thermostat (Lauda eco silver E4, DE) set at 37±0.5° C. The receptor chamber was filled with the dissolution medium and sampled at preset time interval through the side arm of the cell. The analysis was conducted by employing the fine fraction deposited on the diffusion membrane filter after aerosolization by Fast Screening Impactor (FSI). The in vitro aerodynamic assessment was carried following the procedure detailed in the European Pharmacopoeia 10.0 in the 2.9.18 “Preparation for inhalation: Aerodynamic assessment of fine particles” chapter at p 347-360. RS01 device was used to conduct the analysis. The capsules used were Quali-V®-I, size 3 (Qualicaps Europe, S.A.U.) and loaded with about 20 mg of composition powder. The inhaler was activated at a pressure drop of 4 kPa corresponding at a flow rate of 65 L/min for a duration of time sufficient to sample an air volume of 4.0 liters. The FSI was connected to the vacuum pump and the airflow was fixed using a flow meter. The analysis was performed under critical flow control conditions. The device was connected to the FSI through a rubber adaptor; and the content of two or four capsules were aerosolized, and two or four fine particle doses were collected into the apparatus for the 800 or 400 μg strength respectively. The analysis was done in triplicate for each selected composition. After aerosolization, the filter was removed by the FSI and located on the RespiCell™, between the donor chamber and the receptor chamber. 1 ml of dissolution medium were applied on the filter to get it completely wet before the analysis. 1 mL of the receiving solution was removed at fixed intervals by the receptor chamber and replaced with 1 mL of fresh dissolution medium after every withdrawal to maintain a constant volume. In order to assess the amount of drug not dissolved or entrapped in the filter, the residual not-dissolved powder was recovered by washing the filter with 10 mL of acetonitrile:water 60:40, at the end of experiment. The amount of drug in the samples was assessed by HPLC. The data were expressed as percentage of the compound of formula (I) dissolved and 100% of the dissolution corresponded to the drug amount dissolved at the end of experiment. The dissolution profiles were examined in terms of fraction and overall amount dissolved over time, using the difference (f1) and similarity factors (f2) already proposed to compare the dissolution profiles of oral dosage forms (Shah, V. P. et al., FDA Guidance for Industry 1 Dissolution Testing of Immediate Release Solid Oral Dosage Forms. Dissolut. Technol. 1997, 4, 15-22; EMA, CHMP, Guideline on the Investigation of Bioequivalence, https://www.ema.europa.eu/en/documents/scientific-guideline/guideline-investigationbioequivalence-rev1_en.pdf).

The difference factor (f1) calculates the percent difference between two dissolution profiles at each time point and is a measurement of the relative error between the two profiles.

The difference factor (f1) is calculated as follows:

$${f}_{1} = ( \frac{{\sum }_{t = 1}^{n} \vert {R}_{t} - {T}_{t} \vert}{{\sum }_{t = 1}^{n} {R}_{t}} ) \times 100$$

The similarity factor (f2) is calculated as follows:

$${f}_{2} = 50 \times log [ \frac{100}{\sqrt{1 + \frac{{\sum }_{t = 1}^{n} {( {R}_{t} - {T}_{t} )}^{2}}{n}}} ]$$

n is the number of time points, Rt is the mean dissolution value for the reference product at time t, and Tt is the mean dissolution value for the test product at that same time point. The evaluation of f1 and/2 is based on the following conditions: a minimum of three time points (zero excluded) should be considered, and the time points should be the same for the two compositions, and not more than one mean value should exceed 85% of the dissolved drug for any of the compositions. In addition, the relative standard deviation (coefficient of variation) should be less than 20% for the first time point and less than 10% for the other time points considered. A difference factor (f1) value lower than 15 (0-15) indicates no significant difference between the dissolution profiles. A similarity factor (f2) value higher than 50 (50-100) indicates similarity between the two dissolution profiles.

Comparative in vitro dissolution tests between the Reference Product and the drug product of the invention at 400 and 800 μg dose were performed, and as it can be appreciated from FIGS. 7 and 8, the profiles turned out to be almost overlappable, within the experimental limits of the method.

Based on these in vitro results and the indications/assumptions of the inhalation Bioclassification System (Hasted J E et al AAPS/FDA/USP Workshop March 16-17^{th}, Baltimore, AAPS Open, 2016, 2(1), 2016), an IVIV correlation model could be set up to demonstrate plausible bioequivalence, and candidates the product as a biowaiver.

### Example 4: Aerodynamic Profile at Different Air Flow-Rate

Current European Pharmacopoeia (Ph. Eur.) and United States Pharmacopeia (USP) compendial procedures for assessing DPI drug delivery performance require that 4.0 L of air at a pressure drop of 4 kPa be drawn through the inhaler to quantify delivered dose uniformity and aerodynamic particle size distribution.

However, according to the Guideline on the Pharmaceutical Quality of Inhalation and Nasal Products, it is recommended that DPIs show consistent delivery performance across a specific range of flow rates/inspiratory effort, which should be representative of what is achievable by the intended patient population.

Hence, the deposition profile and the dose delivery of the Drug Products according to the composition invention at 800 μg was determined at different flow rates. The in vitro aerodynamic assessment was carried out using a Next Generation Impactor (NGI).

RS01 high resistance with code 239700002AA device (Plastiape, Osnago, LC Italy) was used to conduct the analysis. The capsules used were Quali-V®-I, size 3 (Qualicaps Europe, S.A.U.) and loaded with about 20 mg. The DPI inhaler was activated at a pressure drop of 1.5 kPa corresponding at a flow rate of 40 L/min, at a pressure drop of 4 kPa corresponding at a flow rate of 65 L/min and at a pressure drop of 9.5 kPa corresponding at a flow rate of 100 L/min. For each test the duration of time was set to be sufficient to sample an air volume of 4.0 liters.

The procedure of the experiment, the collection of the samples and the calculation of the aerodynamic parameters were done as detailed in Example 1.

TABLE 10
APSD of RS01 device loaded with the composition of the
invention at 800 μg at different air flow rate applied

| Composition of the | | | |
|---|---|---|---|
| invention | 1.5 kPa | 4 kPa | 9.5 kPa |
| (800 μg/20 mg) | (40 L/min) | (65 L/min) | (100 L/min) |
| ED | 463.2 ± 13.2 | 545.4 ± 4.6 | 492.5 ± 10.4 |
| (μg) | (2.8) | (0.8) | (2.1) |
| MMAD | 2.06 ± 0.03 | 2.02 ± 0.07 | 1.90 ± 0.04 |
| (μm) | (1.56) | (3.23) | (2.29) |
| FPM | 279.7 ± 7.9 | 314.0 ± 13.0 | 280.3 ± 21.1 |
| (μg) | (2.8) | (4.1) | (7.5) |
| FPF | 60.4 ± 1.3 | 57.6 ± 2.8 | 56.9 ± 3.1 |
| (%) | (2.1) | (4.9) | (5.5) |

Emitted Dose (μg), MMAD (μm), FPM (μg), FPF (%) mean values of RS01 device loaded with the composition of the invention (800 μg of compound of formula (I)/20 mg) ± St. dev and CV % in (n = 3)

The respiratory patient profile is an issue that must be addressed for an efficient DPI performance. The development of a flow-rate independent DPI is a strategy to overcome this matter. As it can be appreciated from Table 10, the composition of the invention showed a consistent ED, MMAD, FPM and FPF independently of the applied flow rate in the range of 40-100 L/min.

1: A drug product, comprising a single-dose dry powder inhalation device, comprising an inhaler body (2) defining a recess (3) for a capsule (4), wherein the capsule (4) holds herein a pharmaceutical composition to be inhaled, a nosepiece or mouthpiece (5) communicating with the recess (3), at least one rupturing element (7) coupled to the inhaler body (2) and configured for rupturing the capsule (4) to allow an outside airflow to be mixed with the pharmaceutical composition of the capsule (4) and inhaled through the nosepiece or the mouthpiece (5), and
a pharmaceutical composition filled in a capsule, the pharmaceutical composition comprising micronized particles having a size of from 0.1 to 15 microns of the compound of formula (I) 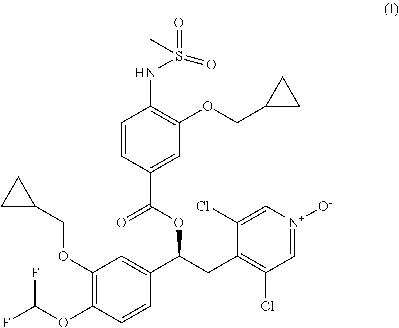 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]
and a carrier consisting of a mixture of coarse and fine particles of pharmaceutically inert acceptable excipient,
wherein the inspiratory flow rate of said inhalation device is from 30 l/min to 65 l/min and
wherein the carrier consists of a mixture of coarse and fine particles of a physiologically acceptable inert excipient in a ratio comprised of from 70:30 to 95:5 by weight,
the coarse particles have an equivalent volume diameter of from 200 to 500 micron,
the fine particles have a dv(0.1) of from 1 to 5 μm, a dv(0.5) of from 18 to 30 μm, and a dv(0.9) of from 65 to 95 μm, as measured by means of laser diffraction or a sieve analyzer.

2: The drug product according to claim 1, wherein the carrier is selected from the group consisting of a polyol, a crystalline sugar, an inorganic salt, an organic salt, an organic compound, a polysaccharide, and an oligosaccharide.

3: The drug product according to claim 1, wherein the coarse particles have an equivalent volume diameter of from 300 to 480 micrometers.

4: The drug product according to claim 3, wherein the coarse particles have an equivalent volume diameter of from 350 to 450 micrometers.

5: The drug product according to claim 1, wherein the inspiratory flow rate is from 40 l/min to 65 l/min.

6: The drug product according to claim 1, wherein the unitary nominal dose of the compound of formula (I) is from 400 to 800 μg.

7: The drug product according to claim 6, wherein the unitary nominal dose of the compound of formula (I) is 400 μg.

8: The drug product according to claim 6, wherein the unitary nominal dose of the compound of formula (I) is 800 μg.

9: A method for the treatment of a respiratory disease, comprising administering to a subject in need thereof an effective amount of a pharmaceutical composition comprising micronized particles having a size of from 0.1 to 15 microns of the compound of formula (I) 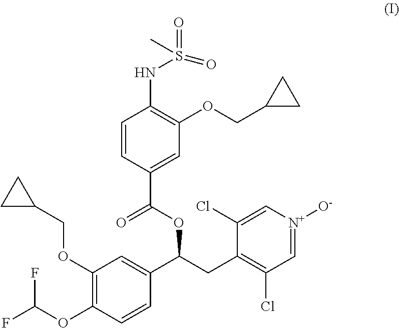 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]
and a carrier for use for the treatment of a respiratory disease,
wherein said composition is administered using a single-dose dry powder inhalation device,
wherein the inspiratory flow rate of said inhalation device is from 30 l/min to 65 l/min, and
wherein the carrier consists of a mixture of coarse and fine particles of a physiologically acceptable inert excipient in a ratio comprised between 70:30 and 95:5 by weight,
the coarse particles have an equivalent volume diameter of from 200 to 500 microns, as measured by means of laser diffraction or a sieve analyzer,
the fine particles have a dv(0.1) of from 1 to 5 μm, a dv(0.5) of from 18 and 30 μm, and a dv(0.9) of from 65 and 95 μm, as measured by means of laser diffraction or a sieve analyzer.

10: The method according to claim 9, wherein the respiratory disease is selected from asthma and chronic obstructive pulmonary disease (COPD).

11: The method according to claim 9, wherein the total daily dose of the compound of formula (I) is from 800 to 4800 μg.

12: The method according to claim 11, wherein the total daily dose of the compound of formula (I) is from 1600 to 3200 μg.

13: A process for the preparation of the drug product according to claim 1, said process comprising:
i) sieving the compound of formula (I) through a suitable mesh,
ii) adding the carrier particles to the compound of formula (I),
iii) sieving the final blend and mixing to obtain the pharmaceutical composition,
iv) filling the obtained pharmaceutical composition in a capsule, and
v) loading the capsule into a medicament chamber of a single dry powder inhalation device.
